(19) United States
(12) Patent Application Publication
(10) Pub. No.: US 2023/0248214 A1
(43) Pub. Date: Aug. 10, 2023

(54) ACTUATORS FOR MEDICAL DEVICES AND RELATED SYSTEMS AND METHODS

(71) Applicants: Boston Scientific Medical Device Limited, Galway (IE); Boston Scientific Scimed, Inc., Maple Grove, MN (US)

(72) Inventors: Aditya DHANOTIYA, Indore (IN); Nabarun BHOWMICK, Kolkata (IN); James WELDON, Newton, MA (US)

(73) Assignees: Boston Scientific Medical Device Limited, Galway (IE); Boston Scientific Scimed, Inc., Maple Grove, MN (US)

(21) Appl. No.: 18/165,569

(22) Filed: Feb. 7, 2023

Related U.S. Application Data
(60) Provisional application No. 63/307,757, filed on Feb. 8, 2022.

(51) Int. Cl. A61B 1/00

(52) U.S. Cl. CPC A61B 1/00068; A61B 1/015

(57) ABSTRACT

According to one aspect, a handle assembly for a medical device may comprise a handle body; and an actuator for controlling a supply of air and a supply of liquid to the medical device. The actuator may comprise a button including a lumen extending through the button; an interior body coupled to the button, wherein a first channel of the interior body is fluidically connected to the lumen; and an exterior body coupled to the handle body, wherein the interior body is received by the exterior body. The actuator is operable at a plurality of different positions along a circumference of the handle body, and the actuator is configured to: supply air to the medical device when a user covers the lumen; and/or supply liquid to the medical device when a user presses the button.

### CROSS REFERENCE TO RELATED APPLIATION(S)

This application claims the benefit of priority of U.S. Provisional Application No. 63/307,757, filed Feb. 8, 2022, the entirety of which is incorporated by reference herein.

### TECHNICAL FIELD

Various aspects of this disclosure relate generally to actuators for handles of medical devices. More specifically, embodiments of this disclosure relate to adjustable actuators for use with a handle of an endoscope or other medical device, among other aspects.

### BACKGROUND

During endoscopic procedures, the medical professional operating the endoscope often wraps his/her entire palm around a grip or handle portion of the device. Various actuators on the handle of the endoscope require the medical professional to contort his/her hands frequently and for prolonged periods of time during a procedure, which can cause strain, or even an injury. In some cases, actuation of different scope controls, like knobs or an elevator, may result in excessive movements of the medical professional’s thumb or other fingers, which may result in strain in the medical professional’s hand. Endoscope operators can experience wrist and hand discomfort resulting from holding and manipulating the endoscope’s handle. In some cases, medical professionals may experience symptoms similar to those of Carpal Tunnel Syndrome or tendonitis. When a medical professional experiences fatigue or other pain in the fingers, hand, or wrist, the medical professional may shift from a primary grip position to a secondary grip position that may be a less powerful grip than the primary grip position, such as shifting from a four finger grip to a three finger grip. Repeatedly reaching or contorting the fingers to access various actuators can increase fatigue or other pain.

When a medical professional repeatedly readjusts his or her hand grip in between procedure tasks, the procedure may be prolonged and procedural tasks may be more difficult. Depending on the size of a medical professional’s hand, actuators may be positioned in non-optimized positions and increase the number of readjustments of the professional’s hand during a procedure.

### SUMMARY

Aspects of the disclosure relate to, among other things, systems, devices, and methods for assisting a user’s access to actuators on a handle and facilitate a user’s hand grip on an endoscope or other medical device. Endoscopes and other medical devices with actuators adjustable to accommodate various sizes of fingers and hands may help address user hand fatigue or strain, may help reduce the need for hand grip readjustments, and may help address other issues. Each of the aspects disclosed herein may include one or more of the features described in connection with any of the other disclosed aspects.

According to one aspect, a handle assembly for a medical device may comprise a handle body; and an actuator for controlling a supply of air and a supply of liquid to the medical device. The actuator may comprise a button including a lumen extending through the button; an interior body coupled to the button, wherein a first channel of the interior body is fluidically connected to the lumen; and an exterior body coupled to the handle body, wherein the interior body is received by the exterior body. The actuator is operable at a plurality of different positions along a circumference of the handle body, and the actuator is configured to: supply air to the medical device when a user covers the lumen; and/or supply liquid to the medical device when a user presses the button.

In other examples, the handle assembly may include one or more of the following features. A body portion of the button may be coupled to the interior body at a rail portion of the interior body, and a recess of the body portion receives the rail portion. The body portion may be U-shaped and includes (i) a first protrusion extending radially-outward from a longitudinal axis of the body portion and (ii) a second protrusion extending radially-outward from a longitudinal axis of the body portion at an opposite side of the body portion as the first protrusion. A control knob may be coupled to the handle body, the interior body may include an opening configured to receive the body portion; and the body portion may be configured to move towards or away from the control knob when the body portion moves from a first side of the opening to a second side of the opening opposing the first side. The interior body may be coupled to the exterior body via at least one spring. A flexible tube may fluidically connect the lumen to the first channel, and the interior body may include a second channel. The interior body may include a recess configured to receive a body portion of the button, the first channel may extend from a first opening into the recess to a second opening in a first side of the interior body, and the second channel extends from a third opening in a second side of the interior body to a fourth opening in the second side, wherein the second side is at an opposite side of interior body from the first side.

In other examples, the handle assembly may include one or more of the following features. A distance from a top surface of the button to a radially-outer edge of the handle body, relative to a central longitudinal axis of the handle body, directly opposite the button may range from 60 to 70 millimeters. The interior body may be configured to move within the exterior body in a direction of a central longitudinal axis of the body portion when a user presses the button to supply liquid to the medical device; and the button may be configured to move relative to the interior body in a direction transverse to the central longitudinal axis. The exterior body may be cylindrical and may be rotatably coupled to the handle body; and the interior body may be cylindrical and coupled to the button. The exterior body may abut an elastic portion of the handle body. The exterior body may include a pair of connector portions projecting radially-outward, relative to a central longitudinal axis, from a radially-outer surface of the exterior body, and each connector portion may be rotatably coupled to the handle body.

In other examples, the handle assembly may include one or more of the following features. The exterior body may include: a fluid inlet; a fluid outlet approximately ninety degrees offset, relative to a central longitudinal axis of the exterior body, from the fluid inlet; an air inlet; and an air outlet approximately ninety degrees offset, relative to a central longitudinal axis of the exterior body, from the air inlet. The exterior body may include: a first recess in a radially-outer surface, relative to a central longitudinal axis, of the exterior body, and a second recess in a radially-outer surface, relative to a central longitudinal axis, of the exterior body, and the second recess may be positioned on an opposite side of the exterior body as the first recess; wherein each of the first recess and the second recess is configured to receive an elastic portion of the handle body. The actuator may be pivotable about a first axis, and the interior body may be movable along a second axis transverse from the first axis.

In other aspects, a handle assembly for a medical device may comprise a handle body; and an actuator for controlling a supply of fluid to the medical device. The actuator may comprise: a button including a lumen extending through the button; an interior body coupled to the button; and an exterior body coupled to the handle body. The interior body may be received by the exterior body; the actuator may be moveable, relative to the handle body, to a plurality of different positions on the handle body, and the actuator may be configured to: supply fluid to the medical device when a user presses the button.

In other examples, the handle assembly may include one or more of the following features. The actuator may be moveable, relative to the handle body, along a first axis; the interior body may be movable, relative to the exterior body, along a second axis transverse from the first axis; and the actuator may be configured to transition from (i) a first operating state in which fluid is not supplied to the medical device to (ii) a second operating state in which fluid is supplied to the medical device when the interior body moves along the second axis. The exterior body may include a fluid inlet; and a fluid outlet approximately ninety degrees offset, relative to a central longitudinal axis of the exterior body, from the fluid inlet; the actuator may be pivotable about a first axis, and the interior body may be movable along a second axis transverse from the first axis.

In other aspects, a method of operating an endoscope that includes a handle is disclosed. The method may comprise: moving a first button from a first position on the handle to a second position on the handle different from the first position; gripping the handle to hold the endoscope; and pressing the first button to supply fluid to the endoscope. The method may further comprise at least one of (a) covering a lumen of the first button to supply air to the endoscope; and (b) moving the first button from the second position on the handle to a third position on the handle different from the first position and the second position.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIGS. 2A and 2B are perspective views of a user’s hand holding an endoscope handle, according to aspects of this disclosure.

FIG. 3 is a top view of an endoscope handle, according to aspects of this disclosure.

FIG. 4 is a side, cross-sectional view of an actuator of the endoscope handle of FIG. 3, according to aspects of this disclosure.

FIG. 5 is a perspective view of the actuator of FIG. 4, according to aspects of this disclosure.

FIG. 6 is a perspective view of a button of the actuator of FIG. 5, according to aspects of this disclosure.

FIG. 7A is a perspective, exploded view of the actuator of FIG. 5, according to aspects of this disclosure.

FIG. 7B is a cross-sectional exploded view of the actuator of FIG. 5, according to aspects of this disclosure.

FIGS. 8A and 8B are partial cross-sectional views of the actuator of FIG. 5, according to aspects of this disclosure.

FIGS. 9A, 9B, 10A, and 10B are perspective, partial cross-sectional views of the actuator of FIG. 5 in various operating states, according to aspects of this disclosure.

FIGS. 11A and 11B are top and bottom views of an endoscope handle including an adjustable actuator, according to aspects of this disclosure.

FIG. 12 is a side view of the adjustable actuator of the endoscope handle of FIGS. 11A and 11B in various positions, according to aspects of this disclosure.

FIG. 13 is an exploded, perspective view of the adjustable actuator of FIG. 12, according to aspects of this disclosure.

FIG. 14A shows a perspective view of a portion of an endoscope handle assembly including adjustable actuators, according to aspects of this disclosure.

FIG. 14B shows a perspective view of a portion of an endoscope handle assembly of FIG. 14A including adjustable actuators with a portion of the endoscope handle removed to expose an interior portion of the endoscope handle, according to aspects of this disclosure.

FIGS. 15A, 15B, and 16 show cross-sectional views of the adjustable actuator of FIG. 12 in different operating states, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve a user’s ability to grip, manipulate, and otherwise apply force to a handle, and actuators of the handle, of a medical device, such as an endoscope, during a medical procedure and, as non-limiting exemplary benefits, help reduce the need to reposition a user’s hand during a procedure and reduce strain to a user’s hand from excessive movement of fingers, among other aspects.

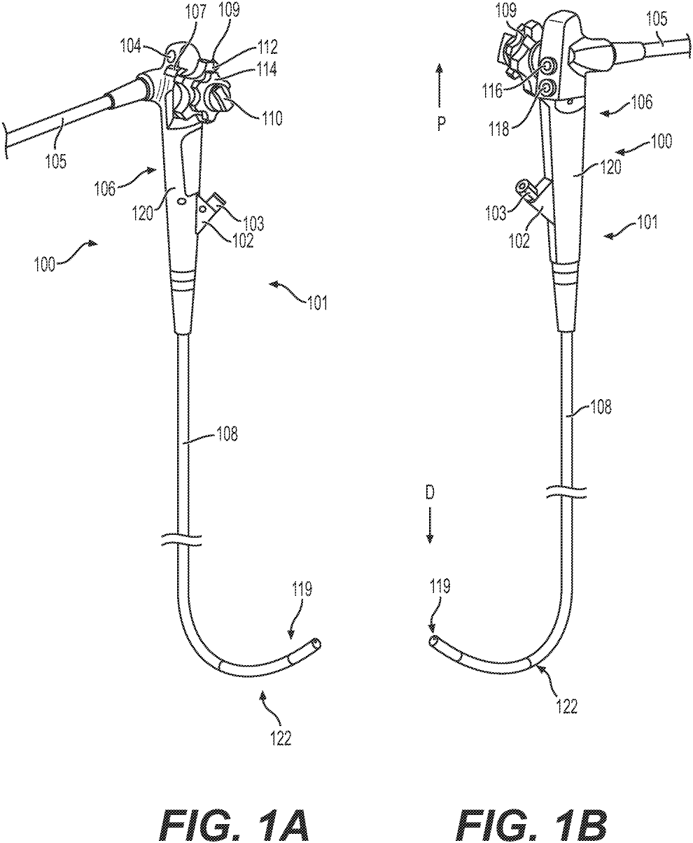

FIGS. 1A and 1B show a perspective view of an exemplary endoscope system 100. Endoscope system 100 may include an endoscope 101. Endoscope 101 may include a handle assembly 106 and a flexible tubular shaft 108. The handle assembly 106 may include a biopsy port 102, a biopsy cap 103, an image capture button 104, an elevator actuator 107, a first locking lever 109, a second locking lever 110, a first control knob 112, a second control knob 114, a suction button 116, an air/water button 118, a handle body 120, and an umbilicus 105. All of the actuators, elevators, knobs, buttons, levers, ports, or caps of endoscope system 100 may serve any purpose and are not limited by any particular use that may be implied by the respective naming of each component used herein. The umbilicus 105 may extend from handle body 120 to auxiliary devices, such as a control unit, water supply, or vacuum source. Umbilicus 105 therefore can transmit signals between endoscope 101 and the control unit, to control lighting and imaging components of endoscope 101 and/or receive image data from endoscope 101. Umbilicus 105 also can provide fluid for irrigation from the water supply and/or suction to a distal tip 119 of shaft 108. Buttons 116 and 118 control valves for suction and fluid supply, respectively. Shaft 108 may terminate at a distal tip 119. Shaft 108 may include an articulation section 122 for deflecting distal tip 119 in up, down, left, and/or right directions. Knobs 112 and 114 may be used for controlling such deflection, and locking levers 109 and 110 may lock knobs 112 and 114, respectively, in desired positions. Handle body 120 may be tapered and may narrow as the handle extends distally such that the profile of the handle body 120 is smaller at its distal end than at its proximal end.

Distal tip 119 may include an imaging device (e.g., a camera) and a lighting source (e.g., an LED or an optical fiber). Distal tip 119 may be side-facing. That is, imaging device and lighting source may face radially outward, perpendicularly, approximately perpendicularly, or otherwise transverse to a longitudinal axis of shaft 108 and distal tip 119.

Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or medical device may be used in connection with the devices of this disclosure, and any of the actuator embodiments discussed herein may be incorporated into any of these or other medical devices.

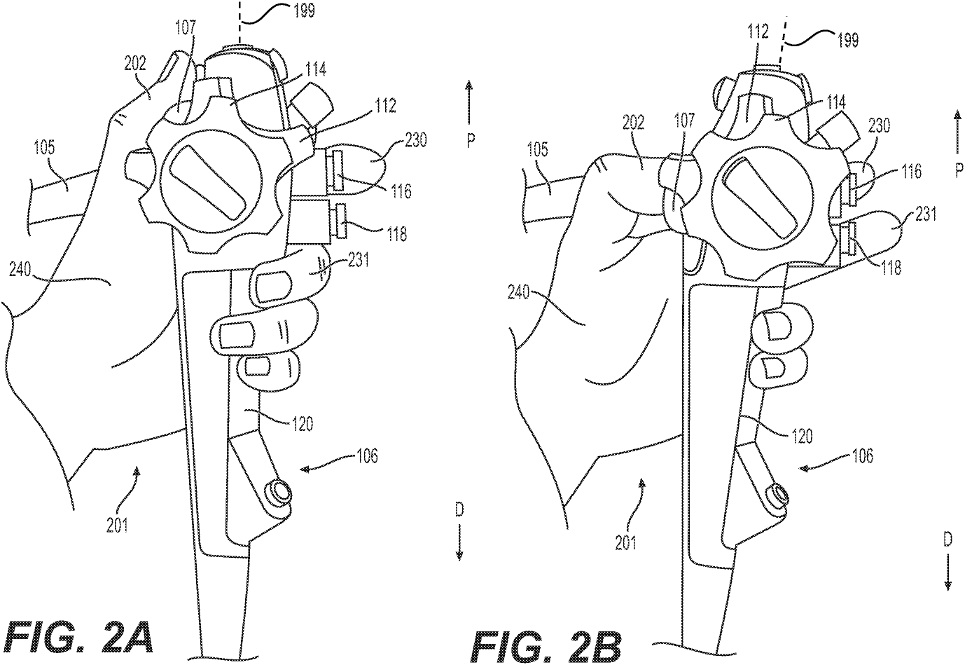

In operating endoscope system 100, a user may use his/her left hand to hold the handle assembly 106 (shown in FIG. 2A) while the right hand is used to hold accessory devices and/or operate one or more of the actuators of the handle assembly 106, such as the first and second control knobs 112, 114 and the first and second locking levers 109, 110. The user may grasp the handle assembly 106 by wrapping the user’s hand around the handle body 120. When grasping handle body 120, the user may use the left thumb to operate the first and second control knobs 112, 114 and the elevator actuator 107 (through rotation about their axis), and may use a left-hand finger to operate the image capture button 104, the suction button 116, and the air/water button 118 (each by pressing).

FIGS. 2A and 2B show an exemplary user’s left hand 201 grasping handle assembly 106. The user’s left index finger 230 and middle finger 231 may be used to operate the suction button 116 and the air/water button 118. A user may have to reach and/or strain their index or middle finger to actuate the suction button 116 and/or the air water button 118. The actuators discussed herein below may help reduce the reach required and/or alleviate strain to a user’s fingers and/or hand. Handle assembly 106 may have a central longitudinal axis 199 extending longitudinally through handle assembly 106.

The user may position the thumb 202 of the grasping hand 201 over the elevator actuator 107 and move the elevator actuator 107 along a circular path from a first position (shown in FIG. 2A) to a second position (shown in FIG. 2B) by moving the thumb 202. As shown in FIG. 2B, the palm 240 may move away from the handle body 120 when the thumb 202 moves from the first position to the second position, and a user may have difficulty reaching the suction button 116 and/or the air water button 118 with the index or middle fingers when the palm 240 moves away from the handle body 120. The actuators discussed herein below may help reduce the movement required of the index and/or ring fingers to actuate the suction button 116 and/or the air water button 118 during operation of endoscope 101.

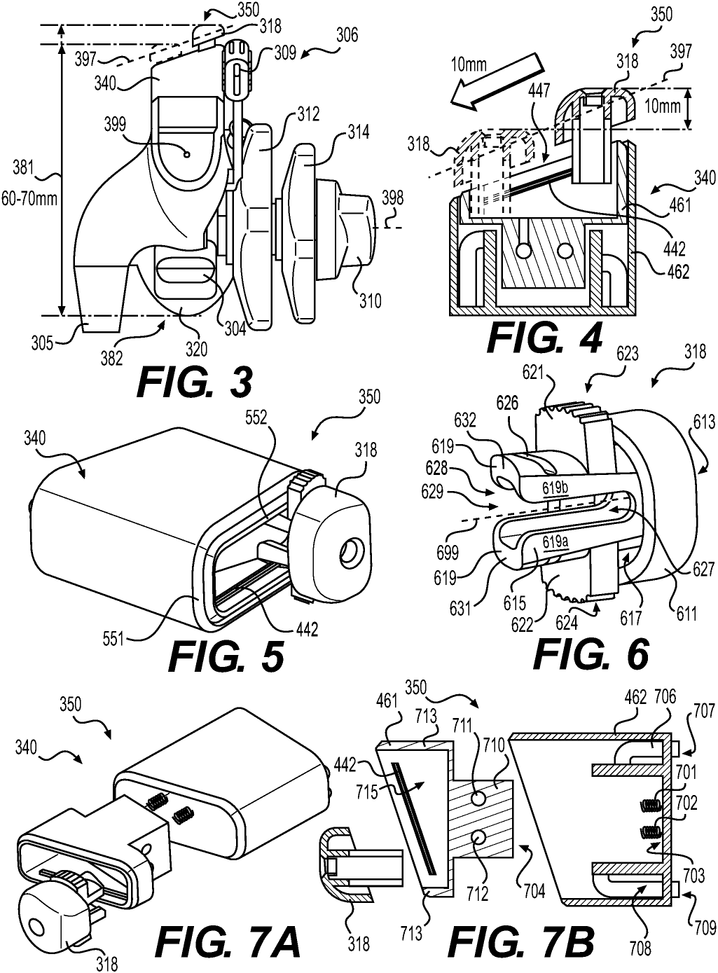

FIG. 3 shows a top view of an alternative handle assembly 306 including an image capture button 304, a first locking lever 309, a second locking lever 310, a first control knob 312, a second control knob 314, an adjustable air/water actuator 350 including air/water button 318 and actuator body 340, a handle body 320, and an umbilicus 305. A central rotational knob axis 398 may extend through first control knob 312 and second control knob 314, and air/water button 318 may be moveable towards or away from first control knob 312 and second control knob 314, and may also be moveable towards or away from the central longitudinal axis 399 of handle assembly 306. Central longitudinal axis 399 is shown extending through the page in FIG. 3. The air/water button 318 may be moveable along an axis 397 transverse from rotational knob axis 398, and axis 397 may not intersect central longitudinal axis 399. Depending on a user’s hand and/or finger size, the user may adjust the positioning of air/water button 318 to allow the user’s fingers to more easily access air/water button 318. For example, by moving air/water button 318 away from first control knob 312 and second control knob 314, and towards umbilicus 305, a user may need to wrap his/her fingers around a smaller portion of the circumference of the handle body 320 to access air/water button 318 when gripping handle body 320. A user may move air water/button 318 to adjust the distance or width 381 from the top of air water/button 318 to the opposite side 382 of handle body 320. In some examples, the width 381 may be adjusted between 60-70 mm (inclusive), as shown in FIG. 3. The attributes discussed herein in relation to air/water button 318 may also be applied to a suction button on a handle assembly of a medical device for controlling actuation of suction applied at a distal portion of the medical device.

FIG. 4 shows a side, cross-sectional view of air water actuator 350 including air/water button 318 and actuator body 340. Actuator body 340 may include an interior moveable body 461 and an exterior fixed body 462. Exterior fixed body 462 may be configured to receive interior moveable body 461, and interior moveable body may include an opening 447 configured to receive air/water button 318. A sliding rail 442 may protrude from an interior surface of interior moveable body 461, and sliding rail 442 may be parallel to axis 397. Further details of actuator body 440 will be discussed in relation to FIGS. 7-10. Top edge portions 551, 552 (shown in FIG. 5) of interior moveable body 461 and an exterior fixed body 462 may be angled and may be substantially parallel to axis 397.

FIG. 5 shows a perspective view of air water actuator 350 including air/water button 318 and actuator body 340. As shown in FIG. 5, sliding rail 442 is a substantially straight, rectangular protrusion and configured to engage with a channel 626 (shown in FIG. 6) of air/water button 318 to hold air/water button 318. Opening 447 may be substantially rectangular, and air/water button 318 may move within opening 447. In some examples, interior moveable body 461 may include two sliding rails 442 on opposing interior surfaces of interior moveable body 461, and each sliding rail 442 may be configured to be received by a channel 626 of air/water button 318.

FIG. 6 shows a perspective view of air/water button 318 removed from actuator body 340. Air/water button 318 may include a cap portion 611 including a central lumen 613, and a body portion 615 coupled to the cap portion 611. Cap portion 611 may be substantially ovular or circular, and may include a cavity or recessed portion 617 that receives the body portion 615. Body portion 615 may include a U-shaped portion 619 and protrusions 621, 622 protruding outwardly from the U-shaped portion 619 at opposite sides of the U-shaped portion 619. Each protrusion 621, 622 may include ribbed portions 623, 624 configured to facilitate a user gripping each protrusion 621, 622. U-shaped portion 619 may include two legs 619a and 619b and a central lumen 629 extending longitudinally through U-shaped portion 619, and two gaps 627, 628 (one in each leg 619a and 619b, respectively) may extend proximally from a distal end 631, 632 of U-shaped portion 619. Legs 619a, 619b of U-shaped portion 619 may be configured to move towards central longitudinal axis 699 of U-shaped portion 619 when a user applies pressure to each protrusion 621, 622, and may be biased towards the position shown in FIG. 6 (the neutral position) such that when a user releases pressure from protrusions 621, 622, legs 619a and 619b of U-shaped portion 619 move back to the neutral position.

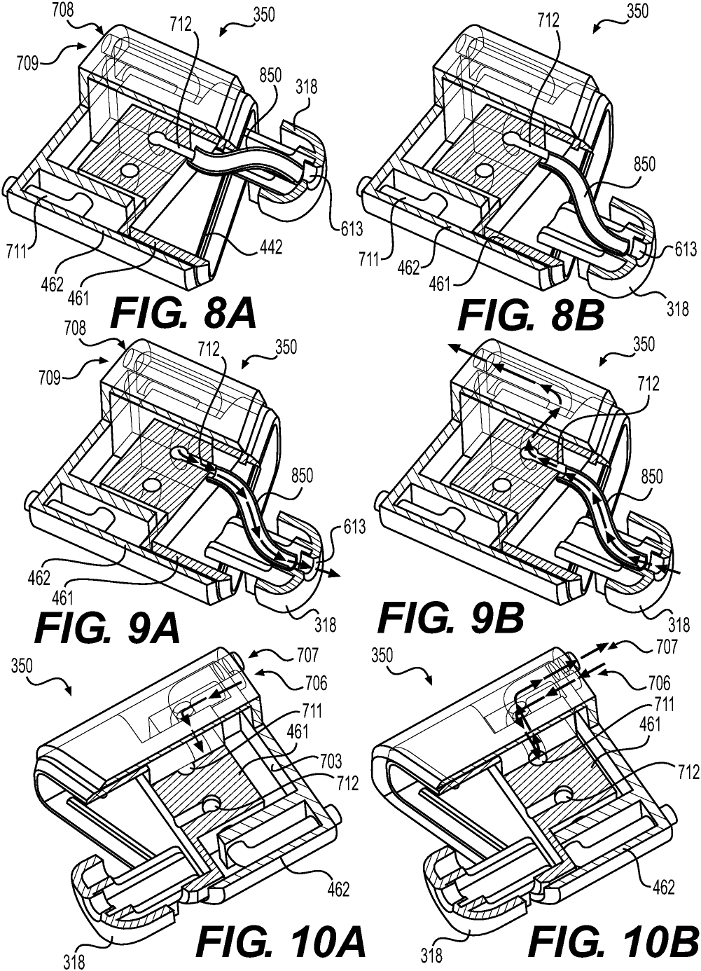

FIGS. 7A and 7B show an exploded view of air water actuator 350 including air/water button 318 and actuator body 340, and a side, cross-sectional, exploded view of air water actuator 350, respectively. As shown in FIGS. 7A and 7B, springs 701, 702 are coupled to a bottom surface 703 of exterior fixed body 462 and a bottom surface 704 of interior moveable body 461. When a user pushes air/water button 318, interior moveable body 461 may move within exterior fixed body 462 and compress springs 701, 702. Exterior fixed body 462 may include a water inlet channel 706 and a water outlet channel 707, and an air inlet channel 708 and an air outlet channel 709. Interior moveable body 461 may include a bottom portion 710 including a water/liquid channel 711 and an air channel 712, and a top portion 713 including a recess 715 and sliding rail 442. As will be discussed further in relation to FIGS. 8-10, water/liquid channel 711 may include two exit openings (not shown) configured to align with the water inlet channel 706 and the water outlet channel 707 of the exterior fixed body 462. The air channel 712 may include a first opening (not shown) configured to align with an opening of the air inlet channel 708, a second opening configured to align with an opening of the air outlet channel 709, and an opening into recess 715.

As shown in FIGS. 8A-8B, air water actuator 350 includes a flexible tube 850 fluidically connecting lumen 613 of air/water button 318 with air channel 712. Tube 850 may be fixedly coupled to an opening of lumen 613 and may form an airtight seal with lumen 613, and may also be fixedly coupled to an opening of air channel 712 and form an airtight seal with air channel 712. Tube 850 may be sized to allow air/water button 318 to move between opposite ends of opening 447 while maintaining a fluidic connection between lumen 613 and air channel 712. Tube 850 is removed in FIGS. 10A-10B for clarity purposes only to facilitate showing fluid flow at various states of air water actuator 350. Tube 850 may move through gaps 627, 628 of body portion 615 to allow for movement of tube 850 without kinking or otherwise disrupting airflow through tube 850.

FIGS. 9A, 9B, 10A, and 10B illustrate air water actuator 350 in various operating states with portions of air water actuator 350 shown in cross-section to show fluid flow paths through the air water actuator 350 at the various operating states. In general, air water actuator 350 allows a user to supply air, or a flow of pressurized air, to a patient’s anatomy through one or more channels of an endoscope or other medical device. When a user leaves lumen 613 of air/water button 318 un-covered, air flow from an air supply is allowed to vent to the atmosphere outside of the medical device and the patient. When a user covers lumen 613 of air/water button 318 with a finger or other part of the user’s body, air flow from the air supply flows through the endoscope or other medical device and exits out of a distal end of the device into the patient’s anatomy. To supply water to the distal end of the endoscope or other device, a user has to press air/water button 318 to supply liquid, such as water, to a water channel of the endoscope or other device. When a user releases air/water button 318, the supply of liquid to the distal end of the endoscope or other device stops. Arrows are shown throughout FIGS. 9A-10B to designate fluid flow paths of liquid or air.

FIG. 9A shows a first state of air water actuator 350 in which neither air nor liquid is supplied to a distal portion of the medical device. In the first state, a user is not covering lumen 613 and is not pressing on air/water button 318. Air flow through air inlet channel 708 flows through air channel 712 and tube 850, and out of lumen 613 in the first state.

FIG. 9B shows a second state of air water actuator 350 when a user covers lumen 613 with a finger or other body member, thus preventing the venting of air out of lumen 613. In the second state, air flow moves through air inlet channel 708, air channel 712, and out of air outlet channel 709 towards a distal end of the endoscope or other medical device. Air may enter tube 850 and flow to, and out of, air outlet channel 709, as shown. In the second state, air is supplied to the patient through an air channel of the endoscope or other medical device. In both the first and second states of air water actuator 350, the user is not pressing on air/water button 318 and springs 701, 702 (not shown in FIGS. 9A and 9B) are in an expanded position.

FIG. 10A shows air water actuator 350 in the first state or second state discussed hereinabove, and shows the fluid flow through water inlet 706. In the first state or second state, liquid (such as water) is not supplied to water outlet channel 707 and not supplied to the patient through the endoscope or other medical device. As shown in FIG. 10A, interior moveable body 461 blocks fluid from water inlet channel 706 from entering into water outlet channel 707, and prevents liquid from being supplied to the endoscope or other medical device. Water/liquid channel 711 is out of alignment with water inlet channel 706, and springs 701, 702 are in an expanded state when water is not supplied to water outlet channel 707.

FIG. 10B shows air water actuator 350 in a third state in which the user is covering lumen 613 and pressing on air/water button 318 to compress springs 701, 702 and move interior moveable body 461 towards bottom surface 703 of exterior fixed body 462. When springs 701, 702 are fully compressed, fluid channel 711 of interior moveable body 461 may be in alignment with water inlet channel 706 and water outlet channel 707, and water/fluid channel 711 may fluidically connect water inlet channel 706 with water outlet channel 707. As shown via the fluid flow arrows in FIG. 10B, liquid is allowed to travel through water inlet channel 706, water/fluid channel 711, and water outlet channel 707 to supply liquid to the patient via the endoscope or other medical device. A user may hold air/water button 318 in the third state to continue to supply liquid to the patient, and then may release air/water button 318 to stop supply of liquid to the patient. When a user releases air/water button 318, air water actuator 350 transitions from the third state to the first or second state (shown in FIG. 10A), and interior moveable body 461 moves through opening 447 via a force from springs 701, 702 transitioning from a compressed state to an expanded state.

In operation, a user may adjust the positioning of air/water button 318 by pinching protrusions 621, 622 and moving air/water button 318 along sliding rail 342 to a position suitable for the user. A user may then proceed with operation of the medical device, and transition air water actuator 350 between the first, second, and third states discussed hereinabove. Air water actuator 350 may be integrated with handle assembly 306 and may be integral with handle body 320, for example if a portion of handle body 320 forms exterior fixed body 462.

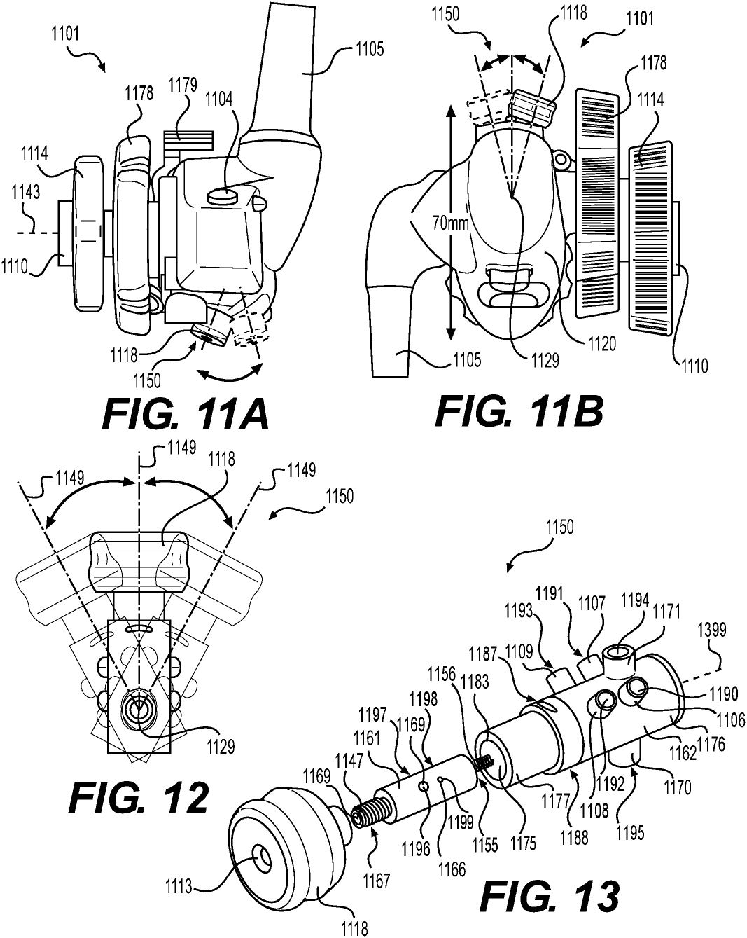

FIGS. 11A and 11B illustrate top and bottom views of a handle assembly 1101 including an image capture button 1104, a first locking lever 1179, a second locking lever 1110, a first control knob 1178, a second control knob 1114, an adjustable air/water actuator 1150 including air/water button 1118, a handle body 1120, and an umbilicus 1105. A central rotational knob axis 1143 may extend through first control knob 1178 and second control knob 1114, and air/water button 1118 may be moveable towards or away from first control knob 1178 and second control knob 1114. Button 1118 may be pivotable about a pivot axis 1129, and in some examples pivot axis 1129 may be aligned with a central longitudinal axis of handle body 1120. In other examples, pivot axis 1129 may be spaced from the central longitudinal axis of handle body 1120. Button 1118 may be configured to pivot such that button 1118 moves substantially along the circumference of handle body 1120, and may be moved to reduce or increase the length required for a user to reach button 1118 with his or her finger. FIG. 12 shows air/water actuator 1150 with button 1118 at various positions, showing how a central longitudinal axis 1149 of button 1118 moves as air/water actuator 1150 is pivoted about pivot axis 1129. In some examples, air/water actuator 1150 may be configured to pivot a total of approximately 30 degrees about pivot axis 1129.

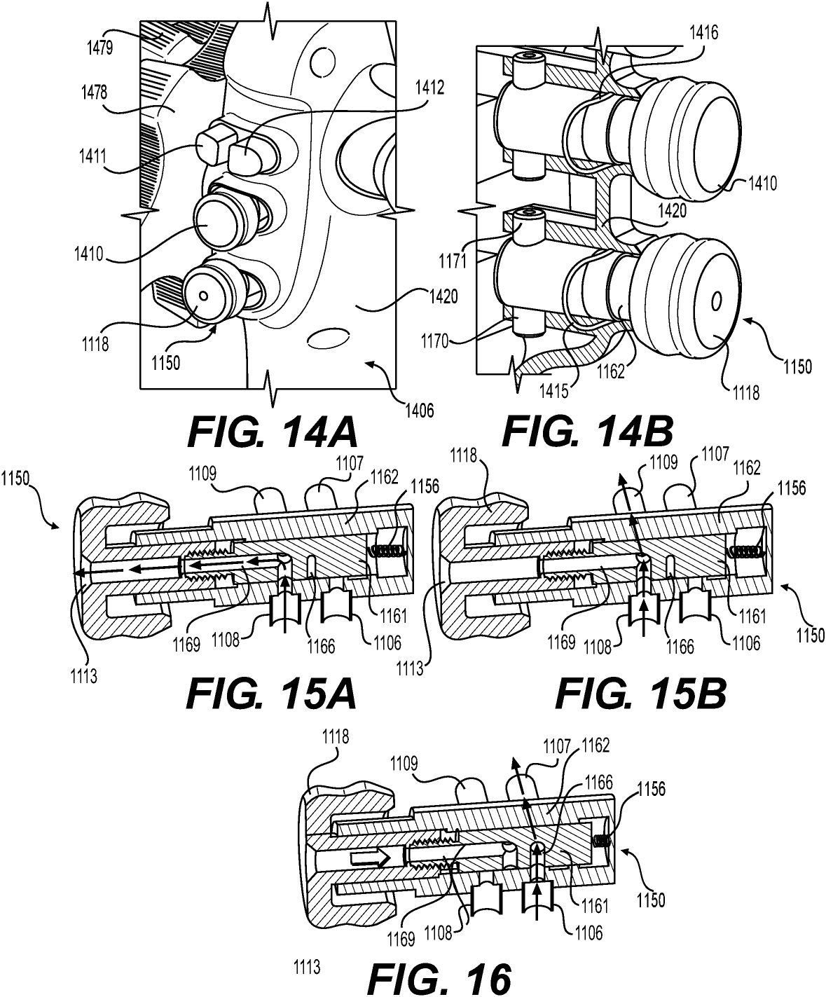

FIG. 13 shows an exploded, perspective view of air/water actuator 1150 including button 1118, interior body 1161, and exterior body 1162. Exterior body 1162 may include a liquid/water inlet 1106, a liquid/water outlet 1107, an air inlet 1108, an air outlet 1109, two connector portions 1170, 1171, a central lumen 1175, main body 1176, protrusion 1177, and a central longitudinal axis 1399. Main body 1176 may be cylindrical, and protrusion 1177 may extend longitudinally outward from a first end of main body 1176. Protrusion 1177 may be cylindrical and have a smaller circumference than main body 1176. Central lumen 1175 may lead to opening 1183 of protrusion 1177 and may be configured to receive interior body 1161. Main body 1176 may include recesses 1187, 1188 on a radially-outer surface, relative to central longitudinal axis 1399, and recesses 1187 may be configured to receive a rubber ring/lining 1415, 1416 (shown in FIG. 14B). In other examples, main body 1176 may not include recesses 1187, 1188.

Liquid/water inlet 1106, liquid/water outlet 1107, air inlet 1108, air outlet 1109, and each of two connector portions 1170, 1171 may be cylindrical and may protrude radially-outward, relative to central longitudinal axis 1399, from main body 1176. In some examples, liquid/water inlet 1106 and liquid/water outlet 1107 may be ninety-degrees offset from each other, relative to longitudinal axis 1399, and air inlet 1108 and air outlet 1109 may be ninety-degrees offset from each other, relative to longitudinal axis 1399. Connector portions 1170, 1171 may be positioned on opposite sides of main body 1176, and may extend radially-outward from central longitudinal axis 1399 of main body 1176 in opposite directions. Liquid/water inlet 1106 and air inlet 1108 may be longitudinally aligned, and liquid/water outlet 1107 and air outlet 1109 may be longitudinally aligned. Connector portion 1171 may be positioned between liquid/water inlet 1106 and liquid/water outlet 1107. Liquid/water inlet 1106, liquid/water outlet 1107, air inlet 1108, and air outlet 1109 may each include a lumen 1190, 1191, 1192, 1193, respectively, and each of lumens 1190-1193 may open into lumen 1175 and be fluidically connected to lumen 1175. Each of connector portions 1170, 1171 may include a recess 1194, 1195, and each recess 1194, 1195 extends towards central longitudinal axis 1399. Each recess 1194, 1195 does not open into lumen 1175.

Interior body 1161 may be cylindrical and configured to be positioned within lumen 1175 of exterior body 1162. Interior body 1161 may include a threaded portion 1167 at a first longitudinal end of interior body 1161, and threaded portion 1167 may be configured to couple to button 1118 (e.g. mate with interior threads of button 1118). Interior body 1161 may include a first tubular, three-pronged, interior channel 1169 with a first prong extending from first opening 1147 at a first end of threaded portion 1167 to an interior portion of interior body 1161; a second prong extending from the first prong to a second opening 1196 at a radially-outer surface, relative to a central longitudinal axis, of interior body 1161; and a third prong extending form the first prong to a third opening 1197 at a radially-outer surface, relative to a central longitudinal axis, of interior body 1161. When interior body 1161 is coupled to button 1118, first interior channel 1169 is fluidically connected to lumen 1113. Interior body 1161 may further include a second interior channel 1166 extending through interior body 1161 from a fourth opening 1198 on a radially-outer surface, relative to a central longitudinal axis, of interior body 1161 to a fifth opening 1199 on a radially-outer surface, relative to a central longitudinal axis, of interior body 1161. The second opening 1196 and third opening 1197 may be configured to align with lumens 1192, 1193, respectively; and fourth opening 1198 and fifth opening 1199 may be configured to align with lumens 1191, 1190, respectively. The second opening 1196 and the third opening 1197 may be ninety-degrees offset from each other about the circumference of the radially-outer surface of interior body 1161, and fourth opening 1198 and fifth opening 1199 may be ninety-degrees offset from each other about the circumference of the radially-outer surface of interior body 1161. First interior channel 1169 may be configured to fluidically connect air inlet 1108 with air outlet 1109, and second interior channel 1166 may be configured to fluidically connect liquid/water inlet 1106 with liquid/water outlet 1107. When air/water actuator 1150 is fully assembled, a spring 1156 may couple a second end 1155 of interior body 1161 to an interior portion of exterior body 1162, such as a bottom interior surface of body 1162.

FIG. 14A shows an exemplary portion of a handle assembly 1406 including an image capture button 1411, general actuator 1412, a first control knob 1478, a second control knob 1479, an adjustable suction actuator 1410, adjustable air/water actuator 1150 including air/water button 1118, and a handle body 1420. Air/water button 1118 is positioned adjacent to first control knob 1478 at a proximal portion of handle body 1420. FIG. 14B illustrates a partial cross-sectional view of handle body 1420, adjustable suction actuator 1410, and adjustable air/water actuator 1150. Each connector portion 1170, 1171 of air/water actuator 1150 is rotatably coupled to handle body 1420. A rubber ring 1415 and/or other rubber lining is coupled to a portion of handle body 1420 and configured to receive exterior body 1162. Rubber ring 1415 may be configured to prevent movement of button 1118 during operation of handle assembly 1406. For example, rubber ring 1415 may be configured to hold exterior body 1162 in place while the force of a single finger is applied to button 1118. A user may move the position of button 1118 by applying a threshold amount of force to force exterior body 1162 to slide across rubber ring 1415 as air/water actuator 1150 pivots about connector portions 1170, 1171. Suction actuator 1410 may have any of the features discussed herein in relation to air/water actuator 1150, including similar structure and function to deliver suction to the medical device and permit adjustment to positioning of actuator 1410, and rubber ring 1416 may interact with suction actuator 1410 in the same manner rubber ring 1415 interacts with air/water actuator 1150.

In discussing FIGS. 15A, 15B, and 16, a medical device is referred to as the medical device in which air/water actuator 1150 is connected to, such as an endoscope or other medical device including handle assembly 1406. FIG. 15A illustrates air/water actuator 1150 in a first operating state in which neither air pressure nor fluid is supplied to the medical device. Flow arrows through air inlet 1108, first interior channel 1169, and lumen 1113 show the path of air flow when a user does not cover lumen 1113 with a finger or other portion of their body.

To transition from the first operating state to the second operating state shown in FIG. 15B, a user must cover lumen 1113 with a finger or other portion of their body to prevent air from exiting out of lumen 1113. As shown by the flow arrows in FIG. 15B, air flow moves through air inlet 1108, first interior channel 1169, and out of air outlet 1109 to be supplied to the medical device when air/water actuator 1150 is in the second operating state. In both the first operating state and the second operating state, the user does not apply pressure to button 1118 and spring 1156 is in an expanded (steady-state, unloaded) position.

FIG. 16 illustrates air/water actuator 1150 in a third operating state in which fluid is supplied to the medical device. Flow arrows through liquid/water inlet 1106, second interior channel 1166, and fluid/water outlet 1107 show the path of liquid/water flow when a user presses, or applies pressure to, button 1118, with a finger or other portion of his/her body such that spring 1156 transitions to a fully compressed (loaded) state. When a user pushes on button 1118, interior body 1161 moves through exterior body 1162, first interior channel 1169 moves out of alignment with air inlet 1108 and air outlet 1109, and second interior channel 1166 moves into alignment with liquid/water inlet 1106 and liquid/water outlet 1107 allowing fluid to flow from inlet 1106 to outlet 1107. When a user releases button 1118, air/water actuator 1150 transitions from the third operating state to the first operating state.

Handle assemblies 306, 1101, 1406 and actuators 350, 1150 of this disclosure may assist with ergonomically positioning fingers of the user when the user operates endoscope 101 or other medical devices, may reduce hand strain caused by excessive movement and/or reaching of fingers when the user operates endoscope 101, and may reduce the chance of the user losing his or her grip. Also, the handle assemblies 306, 1101, 1406 and actuators 350, 1150 may help prevent repeated repositioning of a user’s hand on a medical device handle due to fatigue, strain, or other difficulty. Each of the aforementioned handle assemblies 306, 1101, 1406 and actuators 350, 1150, whether used in conjunction with an endoscope system or any other medical device, may be used to enhance and/or facilitate a user’s grip on a handle. Any portion of the handle assemblies or actuators discussed herein may be incorporated into a handle of an endoscope or other medical device to improve a user’s operation of the device. Handle assemblies 306, 1101, 1406 and actuators 350, 1150 of this disclosure may allow multiple users with different size hands and/or fingers to comfortably use the same handle assembly.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A handle assembly for a medical device comprising:
a handle body; and
an actuator for controlling a supply of air and a supply of liquid to the medical device, the actuator comprising:
a button including a lumen extending through the button;
an interior body coupled to the button, wherein a first channel of the interior body is fluidically connected to the lumen; and
an exterior body coupled to the handle body, wherein the interior body is received by the exterior body;
wherein the actuator is operable at a plurality of different positions along a circumference of the handle body, and the actuator is configured to:
supply air to the medical device when a user covers the lumen; and/or
supply liquid to the medical device when a user presses the button.

2. The handle assembly of claim 1, wherein a body portion of the button is coupled to the interior body at a rail portion of the interior body, wherein a recess of the body portion receives the rail portion.

3. The handle assembly of claim 2, wherein the body portion is U-shaped and includes (i) a first protrusion extending radially-outward from a longitudinal axis of the body portion and (ii) a second protrusion extending radially-outward from a longitudinal axis of the body portion at an opposite side of the body portion as the first protrusion.

4. The handle assembly of claim 2, further comprising a control knob coupled to the handle body, wherein the interior body includes an opening configured to receive the body portion; and wherein the body portion is configured to move towards or away from the control knob when the body portion moves from a first side of the opening to a second side of the opening opposing the first side.

5. The handle assembly of claim 1, wherein the interior body is coupled to the exterior body via at least one spring.

6. The handle assembly of claim 1, wherein a flexible tube fluidically connects the lumen to the first channel, and wherein the interior body includes a second channel.

7. The handle assembly of claim 6, wherein:
the interior body includes a recess configured to receive a body portion of the button,
the first channel extends from a first opening into the recess to a second opening in a first side of the interior body, and
the second channel extends from a third opening in a second side of the interior body to a fourth opening in the second side, wherein the second side is at an opposite side of interior body from the first side.

8. The handle assembly of claim 1, wherein a distance from a top surface of the button to a radially-outer edge of the handle body, relative to a central longitudinal axis of the handle body, directly opposite the button ranges from 60 to 70 millimeters.

9. The handle assembly of claim 2, wherein the interior body is configured to move within the exterior body in a direction of a central longitudinal axis of the body portion when a user presses the button to supply liquid to the medical device;
wherein the button is configured to move relative to the interior body in a direction transverse to the central longitudinal axis.

10. The handle assembly of claim 1, wherein the exterior body is cylindrical and is rotatably coupled to the handle body;
wherein the interior body is cylindrical and coupled to the button.

11. The handle assembly of claim 10, wherein the exterior body abuts an elastic portion of the handle body.

12. The handle assembly of claim 10, wherein the exterior body includes a pair of connector portions projecting radially-outward, relative to a central longitudinal axis, from a radially-outer surface of the exterior body, and each connector portion is rotatably coupled to the handle body.

13. The handle assembly of claim 10, wherein the exterior body includes:
a fluid inlet;
a fluid outlet approximately ninety degrees offset, relative to a central longitudinal axis of the exterior body, from the fluid inlet;
an air inlet; and
an air outlet approximately ninety degrees offset, relative to a central longitudinal axis of the exterior body, from the air inlet.

14. The handle assembly of claim 10, wherein the exterior body includes:
a first recess in a radially-outer surface, relative to a central longitudinal axis, of the exterior body, and
a second recess in a radially-outer surface, relative to a central longitudinal axis, of the exterior body, wherein the second recess is positioned on an opposite side of the exterior body as the first recess;
wherein each of the first recess and the second recess is configured to receive an elastic portion of the handle body.

15. The handle assembly of claim 10, wherein the actuator is pivotable about a first axis, and wherein the interior body is movable along a second axis transverse from the first axis.

16. A handle assembly for a medical device comprising:
a handle body; and
an actuator for controlling a supply of fluid to the medical device, the actuator comprising:
a button including a lumen extending through the button;
an interior body coupled to the button; and
an exterior body coupled to the handle body, wherein the interior body is received by the exterior body;
wherein the actuator is moveable, relative to the handle body, to a plurality of different positions on the handle body, and the actuator is configured to:
supply fluid to the medical device when a user presses the button.

17. The handle assembly of claim 16, wherein the actuator is moveable, relative to the handle body, along a first axis;
wherein the interior body is movable, relative to the exterior body, along a second axis transverse from the first axis; and
wherein the actuator is configured to transition from (i) a first operating state in which fluid is not supplied to the medical device to (ii) a second operating state in which fluid is supplied to the medical device when the interior body moves along the second axis.

18. The handle assembly of claim 16, wherein the exterior body includes:
a fluid inlet; and
a fluid outlet approximately ninety degrees offset, relative to a central longitudinal axis of the exterior body, from the fluid inlet;
wherein the actuator is pivotable about a first axis, and wherein the interior body is movable along a second axis transverse from the first axis.

19. A method of operating an endoscope that includes a handle, the method comprising:
moving a first button from a first position on the handle to a second position on the handle different from the first position;
gripping the handle to hold the endoscope; and
pressing the first button to supply fluid to the endoscope.

20. The method of claim 19, further comprising at least one of (a) covering a lumen of the first button to supply air to the endoscope; and (b) moving the first button from the second position on the handle to a third position on the handle different from the first position and the second position.
